(19) United States
(12) Patent Application Publication
(10) Pub. No.: US 2004/0223935 A1
(43) Pub. Date: Nov. 11, 2004

(54) Cosmetic composition containing a fatty acid glyceride, an alcohol and a silicone emulsifier

(75) Inventor: Christine Meunier, Le Plessis Robinson (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/812,956

(22) Filed: Mar. 31, 2004

Related U.S. Application Data
(60) Provisional application No. 60/463,735, filed on Apr. 18, 2003.

(30) Foreign Application Priority Data
Apr. 9, 2003 (FR) 0304399

(51) Int. Cl. A61K 7/06; A61K 7/11

(52) U.S. Cl. 424/70.12

(57) ABSTRACT

The invention relates to a composition preferably in the form of a water-in-oil (W/O) emulsion containing a fatty acid glyceride, a monoalcohol and a particular silicone surfactant.

### REFERENCE TO PRIOR APPLICATIONS

This application claims priority to U.S. provisional application 60/463,735 filed Apr. 18, 2003, and to French patent application 0304399 filed Apr. 9, 2003, both incorporated herein by reference.

### FIELD OF THE INVENTION

The invention relates to a composition preferably in the form of a water-in-oil (W/O) emulsion containing a fatty acid glyceride, a monoalcohol and a particular silicone surfactant. This composition can be used in particular in cosmetics and/or dermatology.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is common practice to use cosmetic or dermatological compositions consisting of a water-in-oil (W/O) emulsion comprising an aqueous phase dispersed in an oily phase. These emulsions comprise an oily continuous phase and thus make it possible to form a lipid film at the surface of the skin, the consequence of which is to prevent transepidermal water loss and to protect the skin against external attack. These emulsions are thus particularly suitable for protecting and nourishing the skin, but also for any conventional application of cosmetics, which will depend on the active agents contained in the compositions.

However, creams in the form of W/O emulsions have the drawback of giving a fairly greasy feel when applied to the skin, since the oily phase is the external phase. Thus, these creams are generally used for dry skin, since they are too greasy to be used for greasy skin. Furthermore, W/O emulsions do not provide any sensation of freshness and are generally too rich in oils to be used during summer or in countries with a hot climate.

To overcome these drawbacks, it has been envisaged to prepare emulsions with a high alcohol content. Alongside the supply of freshness, these emulsions have additional advantages, for instance better penetration into the skin and a much more gelled and transparent appearance.

However, the alcohol content cannot be too high for reasons of tolerance that may result in skin irritation problems especially in the case of individuals with sensitive skin. Furthermore, the presence of a large amount of alcohol may give rise to additional problems in terms of stability or incompatibility with other commonly used raw materials. This is the case in particular with certain surfactants or emollients such as fatty acid glycerides, or derivatives thereof. The latter agents are used either for their intrinsic characteristics or because they are present in numerous commercial raw materials as solvent. It then becomes difficult to prepare stable W/O emulsions containing these raw materials. In the presence of a large amount of ethanol, the emulsions thus obtained show mediocre stability over time and occasionally even have a tendency to undergo phase separation immediately after they have been manufactured.

### DETAILED DESCRIPTION OF THE PREFERRED EMBODIMENTS

The inventor has discovered, fortuitously, that the use of a particular silicone emulsifier makes it possible to overcome these drawbacks believed to be caused by a large amount of monoalcohol in the presence of certain surfactants or emollients, and to obtain compositions in the form of water-in-oil emulsions that are stable over time.

One subject of the present invention is thus a cosmetic composition comprising:

(a) at least one aqueous phase dispersed in a fatty phase,

(b) at least one glyceride of a C₆ to C₂₂ fatty acid or of a mixture of C₆ to C₂₂ fatty acids, which is optionally polyoxyethylenated and/or polyoxypropylenated,

(c) at least one C₂-C₃ monoalcohol,

(d) at least one silicone emulsifier of formula (I):

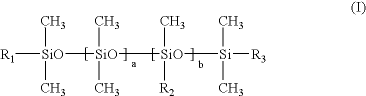

in which:

a is an integer of 0-400,

b is an integer of 0-50, and

a and b cannot simultaneously be equal to 0,

R₁, R₂ and R₃ independently represent a C₁-C₆ alkyl radical or a radical

—(CH₂)ₓ-(OCH₂CH₂)_{y}-(OCH₂CH₂CH₂)_{z}-OR₄, at least one of the radicals R₁, R₂ and R₃ being other than an alkyl radical,

R₄ represents a hydrogen atom, a C₁-C₃ alkyl radical or a C₂-C₄ acyl radical,

x is an integer of 0-6,

y is an integer of 1-30,

z is an integer of 0-30.

According to one preferred embodiment of the invention, in the compound of formula (I), R₁ and R₃ each represent a methyl radical and R₂ represents a radical —(CH₂)ₓ-(OCH₂CH₂)_{y}-(OCH₂CH₂CH₂)_{z}-OR₄ in which a is an integer ranging from 300 to 400, b is an integer ranging from 1 to 10, and y and z are integers ranging from 10 to 20, chosen independently. In these compounds, R₄ will preferably be a hydrogen atom and x an integer ranging from 2 to 4.

As compounds of formula (I) that are particularly suitable for use in the present invention, mention may be made of the dimethicone copolyols sold under the names DC 5329, DC 7439-146 and DC 2-5695 by the company Dow Corning; KF-6013, KF-6015, KF-6016 and KF-6017 by the company Shin-Etsu.

The compounds DC 5329, DC 7439-146 and DC 2-5695 are compounds of formula (I) in which R₁ and R₃ each represent a CH₃ group, R₂ represents a radical —(CH₂)ₓ-(OCH₂CH₂)_{y}-(OCH₂CH₂CH₂)_{z}-OR₄ in which x is 2, z is 0, R₄ is H and, respectively, a is 22, b is 2 and y is 12; a is 103, b is 10 and y is 12; a is 27, b is 3 and y is 12.

More particularly, for the implementation of the present invention, a compound of formula (II):

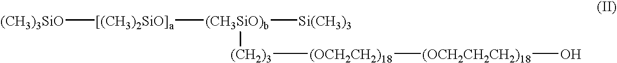

in which a is an integer ranging from 300 to 400 and b is an integer ranging from 1 to 10, and preferably a is 394 and b is 4, is preferred.

The silicone emulsifier may advantageously be present in the composition according to the invention in an amount of between 0.1% and 5% by weight and preferably in an amount of between 0.5% and 3% by weight relative to the total weight of the composition.

According to another embodiment, the silicone emulsifier of formula (I) is combined with a cyclomethicone of formula (III):

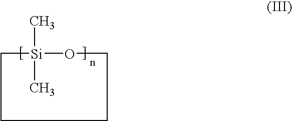

in which n is an integer between 3 and 8.

Mention may be made in particular of cyclotetrasiloxane (n=4), cyclopentasiloxane (n=5) and cyclohexasiloxane (n=6). Cyclopentasiloxane is a silicone that is particularly suitable for implementing the present invention.

The cyclomethicone may advantageously be present in an amount ranging from 5% to 40% by weight and preferably from 10% to 30% by weight relative to the total weight of the composition.

The ratio between the silicone emulsifier of formula (I) and the cyclomethicone preferably ranges between 0.0025 and 1 and more particularly between 0.016 and 0.3.

Included among the silicone emulsifier/cyclomethicone combinations that may be mentioned are:

a mixture of compound of formula (II), cyclotetrasiloxane and water (10/88/2 weight ratio), sold under the name DC 3225C by the company Dow Corning;

a mixture of compound of formula (II), cyclopentasiloxane and water (10/88/2 weight ratio), sold under the name DC2 5225C by the company Dow Corning;

a mixture of compound of formula (II) and cyclopentasiloxane (43/57 weight ratio), sold under the name DC 5185C by the company Dow Corning.

According to one particular embodiment of the invention, the silicone emulsifier of formula (I)/cyclomethicone mixture is the sole emulsifying system of the formulation.

In the context of the present invention, the term “glyceride of a fatty acid” denotes a mixture of monoester, diester and triester of glycerol and of fatty acid, and the term “glyceride of a mixture of fatty acids” means a mixture of monoester, diester and triester of glycerol and of a mixture of fatty acids. Single glycerides (e.g., monoester, diester or triester), or mixtures of two glycerides (e.g., monoester and triester), may also be used. The term “at least one glyceride of a C₆ to C₂₂ fatty acid or of a mixture of C₆ to C₂₂ fatty acids” includes all such possibilities.

Preferred polyoxyethylenated and/or polyoxypropylenated glycerides of a fatty acid or of a mixture of fatty acids are in the form of a derivative of poly(ethylene) glycol and/or of poly(propylene) glycol and of a mixture of monoglyceride, diglyceride and triglyceride of a fatty acid or of mixtures of fatty acids.

In one highly preferred aspect of the invention, the optionally polyoxyethylenated and/or polyoxypropylenated fatty acid glycerides are glycerides of a mixture of fatty acids, and more particularly of a mixture of caprylic acid and capric acid. Polyoxyethylenated and/or polyoxypropylenated glycerides of a fatty acid or of a mixture of fatty acids will be more particularly preferred. Derivatives of polyethylene glycol (4 EO or 6 EO) and of a mixture of caprylic and capric acid monoglyceride, diglyceride and triglyceride will especially be chosen. Mention may be made in particular of Glycerox 767 sold by the company Croda.

The amount of optionally polyoxyethylenated and/or polyoxypropylenated glyceride of a fatty acid or of a mixture of fatty acids in the composition according to the invention may advantageously range from 0.001% to 20%, preferably from 0.1% to 10% and more particularly from 0.1% to 5% by weight, relative to the total weight of the composition.

The monoalcohol present in the compositions according to the invention contains 2 or 3 carbon atoms in a straight or branched chain, and may thus be chosen from ethanol, propanol and isopropanol. Ethanol will preferably be chosen. The monoalcohol is advantageously present in an amount that may be up to 50% by weight, preferably in an amount of between 5% and 50% by weight and more particularly between 10% and 40% by weight relative to the total weight of the composition.

Preferably, the composition according to the invention is intended for topical care or treatment. In this case, the emulsion should contain a physiologically acceptable medium, i.e. a medium that is compatible with the skin, mucous membranes, the nails, the scalp and/or the hair. In addition, it preferably contains at least one active agent and finds its application in a large number of cosmetic and/or dermatological treatments of the skin, including the scalp, the hair, the nails and/or mucous membranes, in particular for the care and/or making up of and/or antisun protection of the skin and/or mucous membranes, and also for the preparation of a cream for treating skin diseases.

According to one preferred embodiment, the composition according to the invention contains at least one lipolytic active agent or an agent that has direct or indirect favorable activity on decreasing adipose tissue. Among these derivatives are phosphodiesterase inhibitors, plant extracts and extracts of marine origin, which are either active on the receptors to be inhibited, such as β-2-blockers and NPY-blockers (described in patent EP-0 838 217), or inhibit the synthesis of LDL or VLDL receptors, or are active in stimulating the β receptors and the G proteins, leading to the activation of adenylcyclase, peptides derived from parathyroid hormone as described in patents FR-2 788 058 and FR-2 781 231 from Sederma, or the peptides described in document FR 2 786 693, or any other peptide that has lipolytic properties, and protamines and derivatives thereof such as those described in document FR-2 758 724.

By way of example, mention may be made of xanthine derivatives, for instance caffeine and its derivatives, especially the 1-hydroxyalkylxanthines described in document FR-2 617 401, caffeine citrate, theophylline and its derivatives, theobromine, acefylline, aminophylline, chloroethyltheophylline, diprofylline, diniprophylline, etamiphylline and its derivatives, etofylline and proxyphylline; combinations containing xanthine derivatives, for instance the combination of caffeine and silanol (caffeine methylsilanetriol derivative), and for example the product sold by the company Exsymol under the name caféisilane C; compounds of natural origin containing xanthine bases, and especially caffeine, such as extracts of tea, of coffee, of guarana, of maté, of cola (Cola nitida) and especially the dry extract of guarana fruit (Paulina sorbilis) containing 8% to 10% caffeine; ephedrine and its derivatives which may be found especially in natural form in plants such as Ma Huang (Ephedra plant); plant extracts of Garcinia cambogia, extracts of Bupleurum chinensis, extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, complexes of phospholipid and of proanthocyanidines from common horse chestnut bark, extracts of cangzhu, extracts of Chrysanthellum indicum, sapogenins such as diosgenin or hecogenin, derivatives thereof and natural extracts containing them, in particular wild yam, extracts of plants of the genus Armeniacea, Atractylodis platicodon, Sinom-menum, Pharbitidis or Flemingia, extracts of Coleus such as C. forskohlii, C. blumei, C. esquirolii, C. scutellaroides, C. xanthantus and C. barbatus, such as the extract of the root of Coleus barbatus containing 60% forskolin, extracts of Ballota, extracts of Guioa, of Davallia, of Terminalia, of Barringtonia, of Trema or of Antirobia, extracts of algae or of phytoplankton, such as rhodysterol or the extract of Laminaria digitata sold under the name PHYCOX75 by the company Secma, the alga skeletonema described in patent FR-2 782 921 or the diatoms described in patent FR-2 774 292.

The amount of lipolytic active agent(s) may vary within a wide range and depends on the nature of the active agent(s) used. In general, the slimming active agent(s) is(are) advantageously present in a concentration ranging from 0.001% to 20% and preferably from 0.1% to 10% by weight relative to the total weight of the composition.

In one particular embodiment, the composition according to the invention contains at least one extract of a diosgenin-rich Dioscorea plant, for example obtained from wild yam root. It is possible, for example, to choose an extract of Dioscorea opposita root sold as a solution in a derivative of polyethylene glycol (6EO) and of a mixture of caprylic and capric acid mono-, di- and triglyceride/preserving agents/glycerol (weight ratio 1/93.8/0.2/5), sold under the name Dioschol by the company Sederma.

The composition according to the invention may also contain at least one other active agent such as those conventionally used in cosmetics, for example desquamating agents capable of acting either by promoting exfoliation, or on the enzymes involved in desquamation or the degradation of the corneodesmosomes, moisturizers, depigmenting or propigmenting agents, anti-glycation agents, NO-synthase inhibitors, 5α-reductase inhibitors, lysyl and/or prolyl hydroxylase inhibitors, agents for stimulating the dermal or epidermal synthesis of macromolecules and/or for preventing their degradation, agents for stimulating the proliferation of fibroblasts or keratinocytes and/or for stimulating keratinocyte differentiation, muscle relaxants, antimicrobial agents, tensioning agents, antipollution agents or free-radical scavengers, anti-inflammatory agents, agents acting on the capillary circulation, and agents acting on the energy metabolism of cells.

The active agent may advantageously be present, for example, in a concentration ranging from 0.01% to 20%, preferably from 0.1% to 5% and better still from 0.5% to 3% relative to the total weight of the composition.

The composition of the invention may also contain adjuvants that are common in cosmetics and/or dermatology, such as preserving agents, antioxidants, complexing agents, solvents, fragrances, fillers, screening agents, bactericides, odour absorbers, dyestuffs and lipid vesicles. The amounts of these various adjuvants are those conventionally used in the field under consideration, for example from 0.01% to 20% of the total weight of the composition. Depending on their nature, these adjuvants may be introduced into the fatty phase or into the aqueous phase.

The oily phase of the composition according to the invention may contain, besides the silicone oil optionally mixed with the emulsifier, oils and fatty substances of any kind that are well known to those skilled in the art, for instance oils of plant origin such as apricot kernel oil, oils of animal origin, hydrocarbon-based oils such as isohexadecane, isododecane, isoparaffins, silicone oils, fluoro oils and/or mineral oils, essential oils and fragrances, and also mixtures of these oils.

The oily phase may also contain other fatty constituents, such as fatty alcohols and fatty acids.

The oily phase is advantageously present in the composition according to the invention in an amount ranging from 5% to 40%, preferably from 8% to 22% and more particularly from 12% to 20% by weight relative to the total weight of the composition.

Another advantage of the composition according to the invention arises from the fact that a large amount of electrolyte may be incorporated therein without harming the stability of the composition.

Examples of useful electrolytes include monovalent, divalent or trivalent metal salts and more particularly alkaline-earth metal salts such as barium, calcium and strontium salts; alkali metal salts such as sodium and potassium salts, magnesium, beryllium, yttrium, lanthanum, cerium, praseodymium, neodymium, promethium, samarium, europium, gadolinium, terbium, dysprosium, holmium, erbium, thulium, ytterbium, lutetium, lithium, tin, zinc, manganese, cobalt, nickel, iron, copper, rubidium, aluminium, silicon and selenium salts, and mixtures thereof.

The ions constituting these salts may be chosen, for example, from carbonates, bicarbonates, sulphates, glycerophosphates, borates, chlorides, bromides, nitrates, acetates, hydroxides, persulphates and also the salts of α-hydroxy acids (citrates, tartrates, lactates or malates) or of fruit acids, or alternatively amino acid salts (aspartate, arginate, glycocholate or fumarate).

The electrolyte is preferably a salt chosen from calcium, magnesium and sodium salts, and especially the chlorides and sulphates, in particular magnesium sulphate, or a mixture comprising at least magnesium chloride, potassium, chloride, sodium chloride, calcium chloride and magnesium bromide, the said mixture corresponding to Dead Sea salts.

The content of electrolyte, when it is present, generally ranges from 0.1% to 5% and preferably from 1% to 5% by weight relative to the total weight of the composition.

A subject of the present invention is also a cosmetic process for treating the skin, the hair, the nails, the scalp and/or mucous membranes, wherein a composition as defined above is applied to the skin, the hair, the nails, the scalp and/or mucous membranes.

The present invention also relates to the use of a silicone emulsifier of formula (I) as defined above to stabilize a composition comprising at least one optionally polyoxyethylenated and/or polyoxypropylenated glyceride of a fatty acid or of a mixture of fatty acids, in the presence of a large amount of C₂-C₃ monoalcohol, the monoalcohol possibly representing up to 50% by weight relative to the total weight of the composition. The fatty acid glyceride is more particularly a mixture of mono-, di- and triglyceride of a mixture of caprylic and capric acids, which is optionally polyoxyethylenated and/or polyoxypropylenated, for instance a derivative of polyethylene glycol (6EO) and of a mixture of caprylic and capric acid mono-, di- and triglyceride.

Advantageously, the silicone emulsifier is combined with a cyclomethicone of formula (III):

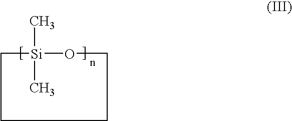

in which n is an integer between 3 and 8.

A combination that is particularly advantageous for implementing the use according to the present invention is the mixture of the compound of formula (II) as defined above, cyclopentasiloxane and water (10/88/2 weight ratio).

As noted above, another preferred embodiment of the invention is use of a silicone emulsifier of formula (I) as defined above, to stabilize a composition comprising an optionally polyoxyethylenated and/or polyoxypropylenated glyceride of a fatty acid or of a mixture of fatty acids, in the presence of an amount of C₂-C₃ monoalcohol ranging up to 50% by weight relative to the total weight of the composition. This use is particularly preferred when the silicone emulsifier is combined with a cyclomethicone of formula (III):

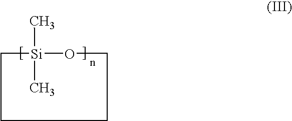

in which n is an integer between 3 and 8, and especially when the emulsifying system is a mixture of compound of formula (II) as defined above, cyclopentasiloxane and water in a 10/88/2 weight ratio and/or when the fatty acid glyceride is a derivative of polyethylene glycol (6EO) and of a mixture of caprylic and capric acid mono-, di- and triglyceride.

The examples that follow illustrate the invention without limiting its scope. Depending on the case, the compounds are cited as chemical names or CTFA names (International Cosmetic Ingredient Dictionary and Handbook).

### EXAMPLE A

### Demonstration of the Stabilization of the Compositions According to the Invention

The aim of this example is to demonstrate the stability of the compositions according to the invention containing the silicone emulsifier of formula (I).

The emulsions of compositions 1 to 4 were prepared in a manner that is conventional for those skilled in the art. The compositions thus obtained were observed in order to evaluate their stability at T=0, and then after 24 hours, 48 hours and 2 months at room temperature, and also after 2 months at 4° C. and at 45° C.

The results are collated in Table 1 (the amounts are expressed as percentages by weight):

TABLE 1

| | Composition | | | |
|---|---|---|---|---|
| | 1 | 2 | 3 | 4 |
| Water | qs 100 | qs 100 | qs 100 | qs 100 |
| Plant extracts | 0.48 | 3.38 | 3.38 | 3.38 |
| Magnesium | 0.7 | 0.7 | 0.7 | 0.7 |
| sulphate | | | | |
| Triethanolamine | | 1.57 | 1.57 | 1.57 |
| Salicylic acid | | 0.5 | 0.5 | 0.5 |
| Glycerol | 12 | 3 | 3 | 3 |
| Polyol | 9 | 6 | 6 | 6 |
| PEG-6 | 2.81 | 2.81 | 3 | |
| caprylic/capric | | | | |
| glycerides (1) | | | | |
| Spring water | 5 | 5 | 5 | 5 |
| Cyclohexasiloxane | 20 | 10 | 10 | 10 |
| Fragrance | 0.3 | 0.3 | 0.3 | 0.3 |
| Ethanol | 20 | | 15 | 15 |
| DC2-5225C (2) | 10 | | | |
| Pemulen TR-2 | | 0.25 | 0.25 | 0.25 |
| (3) | | | | |
| Xanthan | | 0.2 | 0.2 | 0.2 |
| Carbomer (4) | | 0.4 | 0.4 | 0.4 |
| Pecosil PSE (5) | | 2 | 2 | 2 |
| Stability at | Soft | Soft | Destabilization | Soft |
| T = 0 | opaque | opaque | | opaque |
| | gel | gel | | gel |
| Stability at 24 | Identical | Identical | Destabilization | Identical |
| hours | | | | |
| Stability at 48 | Identical | Identical | Destabilization | Identical |
| hours | | | | |
| Stability at 2 | Identical | Identical | Destabilization | Identical |
| months RT | | | | |
| Stability at 2 | Identical | Identical | Destabilization | Identical |
| months 45° C. | | | | |
| Stability at 2 | Identical | Identical | Destabilization | Identical |
| months +4° C. | | | | |

(1) Derivative of polyethylene glycol (6EO) and of a mixture of caprylic and capric acid mono-, di- and triglyceride, sold under the name Glycerox 767 by the company Croda,
(2) DC2 5225 C: mixture of oxyethylenated oxypropylenated (18EO/18PO) polydimethylsiloxane, cyclopentasiloxane and water (10/88/2 weight ratio) sold by the company Dow Corning,
(3) Pemulen TR2: crosslinked acrylic acid/alkyl acrylate copolymer
(4) Carbomer: carboxyvinyl polymer synthesized in an ethyl acetate/cyclohexane mixture,
(5) Pecosil PSE: dimethicone PEG-7 phosphate sold by the company Phoenix Chemical.

The alcohol-free composition is stable (composition 2), as is the composition containing the alcohol without fatty acid glyceride (composition 4). When the alcohol is added to the composition (composition 3), the emulsion is destabilized. The addition of an emulsifier of formula (I) according to the invention (DC2 5225 C in composition 1) produces an emulsion whose stability is comparable to that of compositions 2 and 4.

These results clearly show that the silicone emulsifier of formula (I) makes it possible to stabilize the composition containing a fatty acid glyceride in the presence of monoalcohol. Furthermore, the emulsion thus obtained is stable over time.

### EXAMPLE B

### Comparison of the Stability of the Compositions According to the Invention as a Function of the Emulsifier

The aim of this example is to demonstrate the better stability of the compositions according to the invention containing the silicone emulsifier of formula (I) compared with other known emulsifiers.

Composition 1 (identical to that of composition 1 of Example A) was prepared in a manner that is conventional for those skilled in the art. It comprises the combination of fatty acid glycerides, ethanol and an emulsifier of formula (I) according to the present invention. The composition obtained shows good stability, as shown by the direct observations and also the microscopic observation.

Compositions 5 to 8, prepared in the same manner, contain other conventionally used water-in-silicone emulsifiers. As shown by the results collated in Table 2 below, the emulsions obtained with these emulsifiers are not stable and do not have an appearance that is acceptable on observation (the amounts are expressed as weight percentages):

TABLE 2
Comparison of the stability of emulsions obtained as a function of
the emulsifier

| | Composition | | | | |
|---|---|---|---|---|---|
| | 1 | 5 | 6 | 7 | 8 |
| Water | qs 100 | Qs 100 | qs 100 | qs 100 | qs 100 |
| Polyol | 9 | 9 | 9 | 9 | 9 |
| Magnesium | 0.7 | 0.7 | 0.7 | 0.7 | 0.7 |
| sulphate | | | | | |
| Glycerol | 12 | 12 | 12 | 12 | 12 |
| Plant extracts | 0.48 | 0.48 | 0.48 | 0.48 | 0.48 |
| PEG-6 | 2.81 | 2.81 | 2.81 | 2.81 | 2.81 |
| caprylic/capric | | | | | |
| glycerides (1) | | | | | |
| Spring water | 5 | 5 | 5 | 5 | 5 |
| Cyclopentasiloxane | 20 | 15 | 15 | 15 | 10 |
| Fragrance | 0.3 | 0.3 | 0.3 | 0.3 | 0.3 |
| Ethanol | 20 | 20 | 20 | 20 | 20 |
| DC2-5225C (2) | 10 | | | | |
| ABIL EM 90 (3) | | 1 | | | |
| Xanthan | | | | | 0.2 |
| Carbomer (4) | | | | 0.25 | 0.4 |
| Glucamate | | | 1 | | |
| SSE-20 (5) | | | | | |
| AMPS (6) | | | 0.4 | | |
| Pemulen TR-2 (7) | | | 0.15 | | 0.25 |
| Montanov 68 (8) | | | | 6 | |
| Pecosil PSE (9) | | | | | 2 |
| Neutrol TE | | | | 0.25 | |
| Triethanolamine | | | | | 0.2 |
| Observation after | Nothing to | | Release 6 mm at | Oily edging at | Release 6 mm |
| Centrifugation | report | | the surface | the surface and | at the surface |
| | | | | water drop at | |
| | | | | the bottom | |
| Observation of | Thick | Destabilized | Soft opaque | Soft nacreous | Soft opaque |
| the stability | transparent gel | into 2 phases | gel | milk and | gel |
| | | | | translucent | |
| | | | | edging at the | |
| | | | | surface | |
| Microscopic | Fine tight | | Coarse | Coarse | Coarse |
| observation | emulsion | | emulsion, | emulsion, | emulsion, |
| | without release | | release on the | release on the | release on the |
| | | | edges | edges | edges |

(1) PEG-6 caprylic/capric glycerides: derivative of polyethylene glycol (6EO) and of a mixture of caprylic and capric acid mono-, di- and triglyceride,
(2) DC2 5225 C: mixture of oxyethylenated oxypropylenated (18EO/18PO) polydimethylsiloxane, cyclopentasiloxane and water (10/88/2 weight ratio) sold by the company Dow Corning,
(3) Abil EM 90: cetyldimethicone copolyol sold by the company Dow Corning,
(4) Carbomer: carboxyvinyl polymer synthesized in an ethyl acetate/cyclohexane mixture,
(5) Glucamate SSE-20: PEG-20 methyl glucose sesquistearate sold by the company Amerchol,
(6) AMPS: highly crosslinked polyaminomethylpropane sulphonic acid partially neutralized with ammonia,
(7) Pemulen TR2: crosslinked polymer of acrylic acid/alkyl acrylates,
(8) Montanov 68: cetearyl alcohol and cetearyl glucoside sold by the company SEPPIC,
(9) Pecosil PSE: dimethicone PEG-7 phosphate sold by the company Phoenix Chemical.

### EXAMPLE C

### Comparison of the Stability of the Compositions According to the Invention as a Function of the Amount of Alcohol and of Fatty Acid Triglycerides

Compositions 9 to 11 are prepared in a manner similar to that of the preceding examples (the amounts are expressed as weight percentages). Composition 9 contains 18.7% by weight of fatty acid glycerides. Composition 10 contains 39.45% by weight of ethanol and contains no water. Composition 11 contains 10% by weight of cyclopentasiloxane. Composition 1 is identical to that of Examples A and B.

TABLE 3
Demonstration of the stability of the
emulsifiers as a function of the amount of alcohol

| | Composition | | | |
|---|---|---|---|---|
| | 1 | 9 | 10 | 11 |
| Water | qs 100 | qs 100 | | qs 100 |
| Polyol | 9 | 9 | 9 | 9 |
| Magnesium | 0.7 | 0.7 | 0.7 | 0.7 |
| sulphate | | | | |
| Glycerol | 12 | 12 | 12 | 12 |
| Plant extracts | 0.48 | 1.05 | 0.48 | 0.48 |
| PEG-6 | 2.81 | 18.7 | 2.81 | 2.81 |
| caprylic/capric | | | | |
| glycerides (1) | | | | |
| Spring water | 5 | 5 | 5 | 5 |
| Cyclopentasiloxane | 20 | 20 | 20 | 10 |
| Fragrance | 0.3 | 0.3 | 0.3 | 0.3 |
| Ethanol | 20 | 20 | 39.45 | 20 |
| DC2-5225C (2) | 10 | 10 | 10 | 10 |
| Observation of the | Thick | Thick | Soft | Thick |
| stability | transparent | opaque | opaque | transparent |
| | gel. | gel. | gel. | gel. |
| | Stable | Stable | Stable | Stable |

(1) PEG-6 caprylic/capric glycerides: derivative of polyethylene glycol (6EO) and of a mixture of caprylic and capric acid mono-, di- and triglyceride,
(2) DC2 5225 C: mixture of oxyethylenated oxypropylenated (18EO/18PO) polydimethylsiloxane, cyclopentasiloxane and water (10/88/2 weight ratio) sold by the company Dow Corning.

In each case, the observations after centrifugation and the microscopic observations were found to be satisfactory.

The above results show that the silicone emulsifier of formula (I) according to the present invention makes it possible to obtain a stable composition even when the amount of fatty acid glycerides increases (composition 9) or when the composition contains close to 40% of monoalcohol (composition 10). Furthermore, the amount of cyclomethicone is not a determining factor for the stability of the emulsion (composition 11).

### EXAMPLE D

### Compositions Containing the Emulsifier of Formula (I):

TABLE 4

| | Composition | | | | |
|---|---|---|---|---|---|
| | 12 | 13 | 14 | 15 | 16 |
| Aqueous phase | | | | | |
| Water | qs 100 | qs 100 | qs 100 | qs 100 | qs 100 |
| Plant extract | 0.45 | 0.45 | 0.45 | 0.45 | 0.45 |
| Magnesium | 0.7 | 0.7 | 0.7 | 0.7 | 0.7 |
| Sulphate | | | | | |
| Glycerol | 12 | 12 | 12 | 12 | 12 |
| Butylene glycol | 9 | 9 | 9 | 9 | 9 |
| Dioschol (1) | 3 | 3 | 3 | 3 | 3 |
| Spring water | 5 | 5 | 5 | 5 | 5 |
| Ethanol | 20 | 20 | 20 | 20 | 20 |
| Oily phase | | | | | |
| Cyclopentasiloxane | 10 | 10 | 10 | 10 | |
| Apricot oil | 10 | | | | |
| Isohexadecane | | 10 | | | |
| Isododecane | | | 10 | | |
| Isoparaffin | | | | 10 | |
| Cyclohexasiloxane | | | | | 20 |
| Fragrance | 0.3 | 0.3 | 0.3 | 0.3 | 0.3 |
| DC2-5225C (2) | 10 | 10 | 10 | 10 | 10 |

(1) Dioschol: Extract of Dioscorea opposita (wild yam) root in a mixture: derivative of polyethylene glycol (6EO) and of a mixture of caprylic and capric acid mono-, di- and triglyceride/preserving agents/glycerol (1/93.8/0.2/5 weight ratio) sold by the company Sederma.
(2) DC2 5225 C: mixture of oxyethylenated oxypropylenated (18EO/18PO) polydimethylsiloxane, cyclopentasiloxane and water (10/88/2 weight ratio) sold by the company Dow Corning.

TABLE 5

Composition

| | 17 | 18 |
|---|---|---|
| Aqueous phase | | |
| Water | Qs 100 | qs 100 |
| Caffeine | 3 | 3 |
| Plant extract | 0.2 | 0.2 |
| Niacinamide | | 1 |
| Salicylic acid | 0.72 | 0.72 |
| Magnesium sulphate | 0.7 | 0.7 |
| Trisodium citrate | 2 | |
| Glycerol | 8 | 8 |
| Butylene glycol | 5 | 5 |
| Dioschol (1) | 3 | 3 |
| Spring water | 5 | 5 |
| Ethanol | 20 | 20 |
| Preserving agents | 0.5 | 0.5 |
| Colorants | 0.0001 | 0.0001 |
| Neutralizer | 0.72 | 0.72 |
| Oily phase | | |
| Cyclopentasiloxane | 9 | 9 |
| Isoparaffin | 2 | 2 |
| Cyclohexasiloxane | 5 | 5 |
| Fragrance | 0.3 | 0.3 |
| DC2-5225C (2) | 8 | 8 |

(1) Dioschol: Extract of Dioscorea opposita (wild yam) root in a mixture: derivative of polyethylene glycol (6EO) and of a mixture of caprylic and capric acid mono-, di- and triglyceride/preserving agents/glycerol (1/93.8/0.2/5 weight ratio) sold by the company Sederma.
(2) DC2 5225 C: mixture of oxyethylenated oxypropylenated (18EO/18PO) polydimethylsiloxane, cyclopentasiloxane and water (10/88/2 weight ratio) sold by the company Dow Corning.

Compositions 12 to 18 are prepared in a manner that is standard for those skilled in the art: the aqueous phase and the oily phase are prepared separately without heating. The aqueous phase is then dispersed in the oily phase with vigorous stirring. All the emulsions thus prepared have satisfactory stability conditions on manufacture and over time.

The above written description of the invention provides a manner and process of making and using it such that any person skilled in this art is enabled to make and use the same, this enablement being provided in particular for the subject matter of the appended claims, which make up a part of the original description.

Where used herein the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising:
(a) at least one aqueous phase dispersed in a fatty phase,
(b) at least one glyceride of a C₆ to C₂₂ fatty acid or of a mixture of C₆ to C₂₂ fatty acids, which is optionally polyoxyethylenated and/or polyoxypropylenated,
(c) at least one C₂-C₃ monoalcohol,
(d) at least one silicone emulsifier of formula (I): 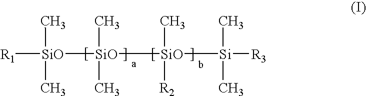
in which:
a is an integer of 0 to 400,
b is an integer of 0 to 50,
a and b cannot simultaneously be equal to 0,
R₁, R₂ and R₃ independently represent a C₁-C₆ alkyl radical or the radical
—(CH₂)ₓ-(OCH₂CH₂)_{y}-(OCH₂CH₂CH₂)_{z}-OR₄, at least one of the radicals R₁, R₂ and R₃ being
—(CH₂)ₓ-(OCH₂CH₂)_{y}-(OCH₂CH₂CH₂)_{z}-OR₄,
R₄ represents a hydrogen atom, a C₁-C₃ alkyl radical or a C₂-C₄ acyl radical,
x is an integer ranging from 0 to 6,
y is an integer ranging from 1 to 30, and
z is an integer ranging from 0 to 30.

2. The composition according to claim 1, wherein, in the compound of formula (I), R₁ and R₃ each represent a methyl radical and R₂ represents a radical —(CH₂)ₓ-(OCH₂CH₂)_{y}-(OCH₂CH₂CH₂)_{z}-OR₄ in which a is an integer of from 300 to 400, b is an integer of from 1 to 10, and y and z independently are integers of from 10 to 20.

3. The composition according to claim 2, wherein R₄ is a hydrogen atom and x is an integer of from 2 to 4.

4. The composition according to claim 1, wherein the silicone emulsifier is represented by formula (II): 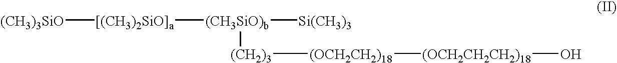
in which a is an integer of from 300 to 400 and b is an integer of from 1 to 10.

5. The composition according to claim 4, wherein, in the compound of formula (II), a is 394 and b is 4.

6. The composition according to claim 1, wherein the silicone emulsifier is present in an amount of 0.1%-5% by weight and preferably in an amount of between 0.5% and 3% by weight relative to the total weight of the composition.

7. The composition according to claim 1, further comprising at least one cyclomethicone of formula (III): 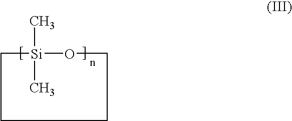
in which n is an integer of 3-8.

8. The composition according to claim 7, wherein the cyclomethicone is selected from the group consisting of cyclotetrasiloxane (n=4), cyclopentasiloxane (n=5), cyclohexasiloxane (n=6) and mixtures thereof.

9. The composition according to claim 7, wherein the at least one cyclomethicone is present in an amount of from 5% to 40% by weight relative to the total weight of the composition.

10. The composition according to claim 7, wherein the ratio between the silicone emulsifier of formula (I) and the cyclomethicone is 0.016-0.3.

11. The composition according to claim 7, comprising the compound of formula (II), cyclopentasiloxane and water in a 10/88/2 weight ratio.

12. The composition according to claim 1, wherein the glyceride of a fatty acid or of a mixture of fatty acids is polyoxyethylenated and/or polyoxypropylenated.

13. The composition according to claim 1, wherein the optionally polyoxyethylenated and/or polyoxypropylenated fatty acid glyceride is a glyceride of a mixture of caprylic and capric acids, which is optionally polyoxyethylenated and/or polyoxypropylenated.

14. The composition according to claim 13, wherein the fatty acid glyceride is a derivative of polyethylene glycol (6EO) and of a mixture of caprylic and capric acid mono-, di- and triglyceride.

15. The composition according to claim 1, wherein the optionally polyoxyethylenated and/or polyoxypropylenated glyceride of a fatty acid or of a mixture of fatty acids is present in an amount of 0.1%-10% by weight relative to the total weight of the composition.

16. Composition according to claim 1, wherein the monoalcohol is present in an amount up to 50% by weight relative to the total weight of the composition.

17. Composition according to claim 1, comprising ethanol.

18. The composition according to claim 1, further comprising at least one lipolytic active agent and/or an agent that has direct or indirect favorable activity on decreasing adipose tissue.

19. The composition according to claim 18, comprising at least one of the following: caffeine, caffeine citrate, theophylline, theobromine, acefylline, aminophylline, chloroethyltheophylline, diprofylline, diniprophylline, etamiphylline, etofylline, proxyphylline, ephedrine, combinations of caffeine and of silanol, extracts of tea, of coffee, of guarana, of mate, of cola (Cola nitida); plant extracts of Garcinia cambogia, extracts of Bupleurum chinensis, extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, complexes of phospholipid and of proanthocyanidines from common horse chestnut bark, extracts of cangzhu, extracts of Chrysanthellum indicum, sapogenins, extracts of plants of the genus Armeniacea, Atractylodis platicodon, Sinom-menum, Pharbitidis or Flemingia, extracts of Coleus such as C. forskohlii, C. blumei, C. esquirolii, C. scutellaroides, C. xanthantus and C. barbatus, extracts of Ballota, extracts of Guioa, of Davallia, of Terminalia, of Barringtonia, of Trema or of Antirobia, extracts of algae or of phytoplankton, extract of Laminaria digitata, the skeletonema alga and diatomaceous earths.

20. The composition according to claim 19, comprising a diosgenin-rich Dioscorea extract derived from wild yam root.

21. The composition according to claim 18, wherein the lipolytic active agent(s) is(are) present in an amount of from 0.1% to 10% by weight relative to the total weight of the composition.

22. The composition according to claim 1, further comprising at least one active agent selected from the group consisting of desquamating agents capable of acting either by promoting exfoliation, or on the enzymes involved in desquamation or the degradation of the corneodesmosomes, moisturizers, depigmenting or propigmenting agents, anti-glycation agents, NO-synthase inhibitors, 5α-reductase inhibitors, lysyl and/or prolyl hydroxylase inhibitors, agents for stimulating the dermal or epidermal synthesis of macromolecules and/or for preventing their degradation, agents for stimulating the proliferation of fibroblasts or keratinocytes and/or for stimulating keratinocyte differentiation, muscle relaxants, antimicrobial agents, tensioning agents, antipollution agents or free-radical scavengers, anti-inflammatory agents, lipolytic active agents or agents that have direct or indirect favorable activity on decreasing adipose tissue, agents acting on the capillary circulation, and agents acting on the energy metabolism of cells.

23. A process for treating the skin, the hair, the nails, the scalp and/or mucous membranes, comprising applying the composition of claim 1 to the skin, the hair, the nails, the scalp and/or mucous membranes.
